(19) United States
(12) Patent Application Publication
(10) Pub. No.: US 2011/0098733 A1
(43) Pub. Date: Apr. 28, 2011

(54) MEDICAL DEVICE ASSEMBLY HAVING FREEDOM OF ROTATION

(76) Inventor: Ky Huynh, Tigard, OR (US)

(21) Appl. No.: 12/606,150

(22) Filed: Oct. 26, 2009

(51) Int. Cl. A61B 17/3211; H01R 4/48; H01R 39/00; A61B 17/00

(52) U.S. Cl. 606/167; 439/816; 439/13; 439/29; 606/1

(57) ABSTRACT

An electrical medical device assembly that includes a handheld unit, adapted to be manipulated by a medical professional and requiring multi-conductor electrical connection to a base unit; a base unit adapted to provide electrical power to the handheld unit; a multi-conductor electrical cable connecting the handheld unit to the base unit; and a multi-conductor electrical connector interposed between the handheld unit and the base unit, the connector including a first half and a second half and wherein relative rotation is permitted between the first half and the second half.

### BACKGROUND

Increasingly, hand held medical devices are connected to a base station with a multi-connector cable ferrying data from the device and commands to the device. Angioplasty catheters, for example, may be coupled with some type of imaging device, such as a simple digital camera or an ultrasound imaging array and may receive command signals. Unfortunately, medical devices must often be manipulated by a medical professional who is concentrating deeply about the task at hand. The manipulation may cause a rotation of the device. As a result the cable for the medical device becomes twisted, resisting further rotation, which may be necessary for a manipulation the health care professional is performing and potentially bending, threatening data and power flow and harming the cable.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a method of performing a surgical procedure using an electrical surgical device that is connected to a base station by a cable having a plurality of mutually electrically insulated conductors, and that has an electrical connector interposed at a point between the base station and the device. The connector has a first half and a second half that, when connected together, have freedom of rotation relative to each other. The surgical procedure is performed and the connector permits the relative rotation, thereby avoiding a problem of cable twisting.

In a second separate aspect, the present invention may take the form of an electrical medical device assembly that includes a handheld unit, adapted to be manipulated by a medical professional and requiring multi-conductor electrical connection to a base unit; a base unit adapted to provide electrical power to the handheld unit; a multi-conductor electrical cable connecting the handheld unit to the base unit; and a multi-conductor electrical connector interposed between the handheld unit and the base unit, the connector including a first half and a second half and wherein relative rotation is permitted between the first half and the second half.

In a third separate aspect, the present invention may take the form of a connector having a first half that defines a plurality of contacts having circular conductive surfaces and a second half that includes a set of resilient contacts, each positioned to contact one of the circular conductive surfaces to create an electrical connection.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is a diagram of a medical device assembly that may be implemented in accordance with the present invention.

FIG. 2A is a top side perspective view of a connector according to the present invention, in closed form.

FIG. 2B is a top side perspective view of the connector of FIG. 1A, in open form.

FIG. 2C is a side sectional view of the connector of FIG. 1A taken along line 1C-1C of FIG. 1A.

FIG. 2D is an alternative preferred embodiment of the connector of the present invention, which is internally the same as the connector of FIG. 1A, but which is embedded into a base station.

FIG. 3A is a top side perspective view of an alternative preferred embodiment of a connector according to the present invention, shown in closed form.

FIG. 3B is a top side perspective view of the connector of FIG. 2A, shown in open form.

FIG. 4A is a top side perspective view of a connector according to an alternative preferred embodiment of the present invention, shown in closed form.

FIG. 4B is a top side perspective view of the connector of FIG. 4A, shown in open form.

### DETAILED DESCRIPTION OF THE PREFERRED EMBODIMENTS

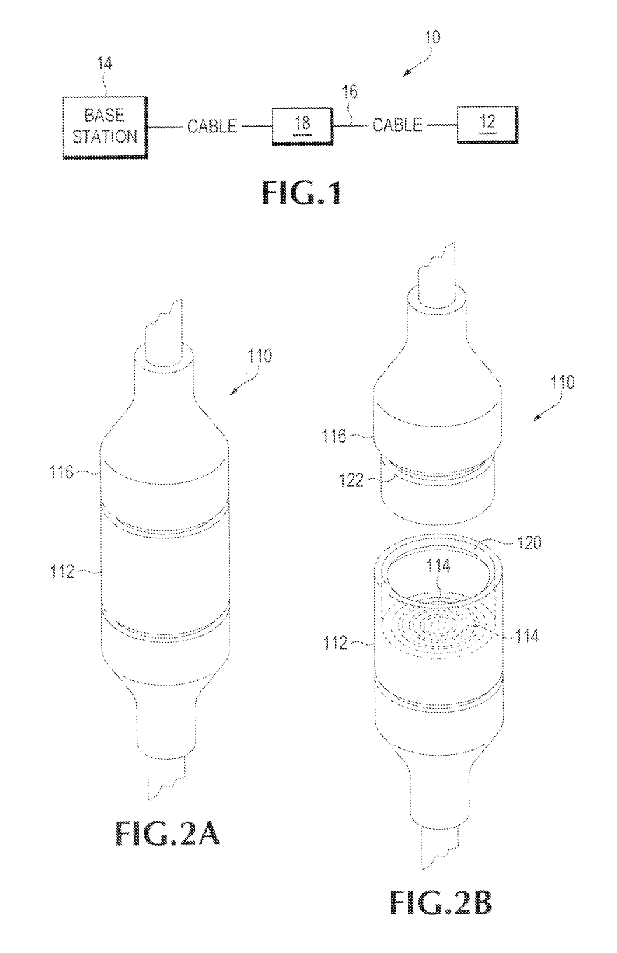

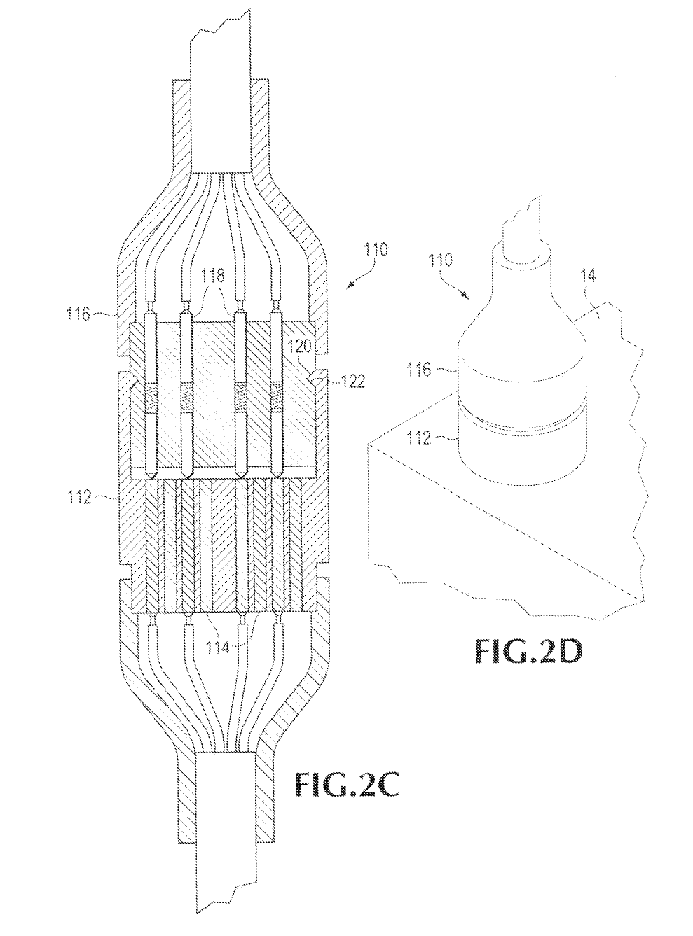

Referring to FIG. 1, a medical device assembly 10 is made up of a handheld medical device 12 connected to a base station 14 by a multi-conductor cable 16. A connector 18 is located either at the point where cable 16 meets base station 14, with one half of the connected being a part of base station 14 (FIG. 2D), or is located between two longitudinal halves of cable 16.

Each one of the following embodiments is shown with only a few contacts for ease of illustration. In reality, however, connectors according to the present invention may have upwards of fifty contacts, which would be necessary to support some of the hand-held devices available today. The contacts are typically have a surface layer of gold plated on nickel, which is plated onto copper. Nickel is used primarily to gain good adhesion of the gold, which does not bond well directly on copper. Gold is used because it does not oxidize. Oxidation could defeat the formation of robustly conductive connection between contacts. Another material that can be used for the contacts is a platinum-iridium alloy.

Referring to FIGS. 2A-2C, in one preferred embodiment a rotatable connector 110 is made up of a first half 112 defining a set of circular contacts 114, arranged concentrically. A second half 116 is made up of a set of spring-loaded, conductive pins 118 (pogo pins, in industry parlance), which are positioned so that each one will touch a circular contact 114 when the first and second halves are joined, thereby forming an electrical connection. when the second half 116 is rotated relative to the first half 112, the pins 118 move in a circle, with each pin maintaining contact with its corresponding circular contact 114. A lip defined by the housing for connector-half 112 fits into a groove 122 in the exterior of connector-half 116, to keep halves 112 and 116 together, but without fitting so tightly as to prevent rotation between the two halves, 112 and 116.

Various techniques may be used in constructing the connector described above. One method of creating concentric circle contacts 114 utilizes conductor deposition techniques used for printed circuit boards. In addition pogo-pins 118, other types of resilient contacts can be made, for example by a wire forming process in which the wire-end is compressed.

In an alternative preferred embodiment (not shown) each circular contact is broken up into a pair of semicircular contacts, with a pin connecting to each one. This alternative embodiment provides twice as many connections, but permits only 180 degree rotation. As noted previously, FIG. 2D shows the case in which half 112 of connector 110 is embedded in base station 14.

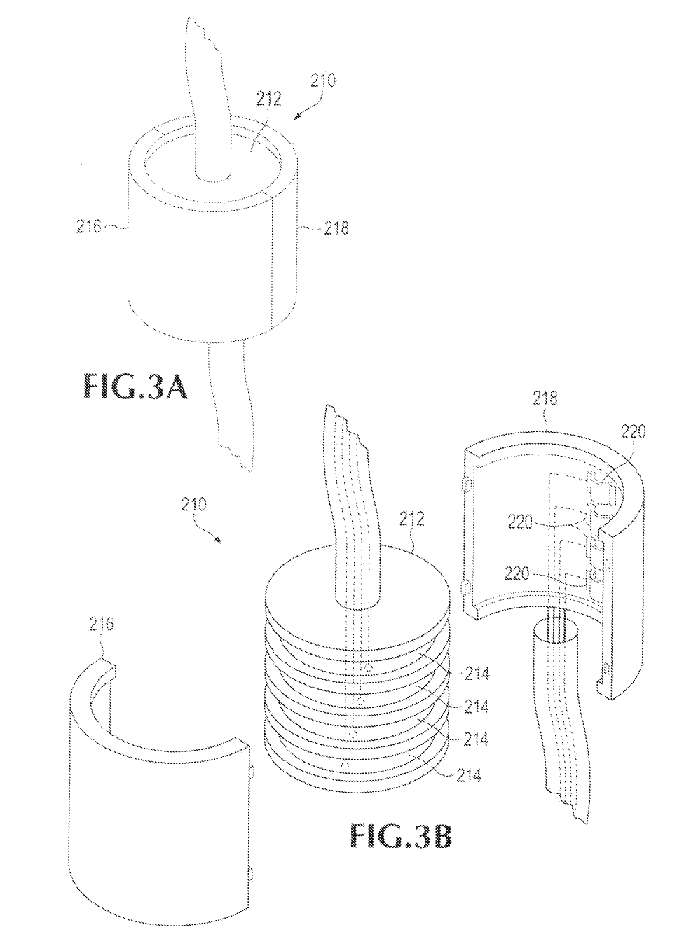

Referring to FIGS. 3A and 3B, in an alternative preferred embodiment of a rotatable multi-contact connector 210, a first half 212 includes a set of circular contacts 214 arranged in stacked form. A second half includes first and a second semi-circular elements 216 and 218, adapted to lock together about first half 212. Element 218 has stacked resilient contacts 220 adapted to touch stacked circular contacts 214.

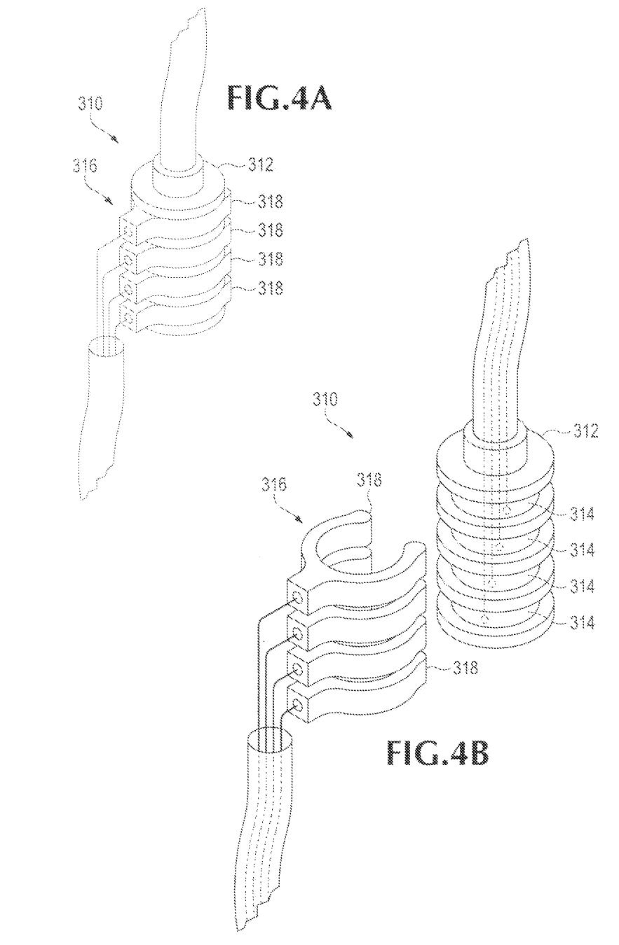

Referring to FIGS. 4A and 4B, a further alternative preferred embodiment of a rotatable multi-conductor connector 310 has a first half 312 similar to first half 212 with stacked circular contacts 314, but wherein a second half 316 has a plurality of resilient horseshoe contacts 318, each being sufficiently flexible to snap about the corresponding circular contact 314. To gain this flexible contacts 318 may be formed of a flexible beryllium copper alloy and may have a thickness of about one millimeter.

In one preferred embodiment circular contacts 214 and/or 314 are made in modular fashion so that they can be easily fit together to form a connector having as many contacts as is desired.

One type of problem potentially encountered by the above described systems is that of a distortion of delicate analog signals caused by a variation in the robustness of the connection between two corresponding contacts whether a pin 118 with a circular contact 114, or a resilient contact 220 with a circular contact 214. One method of addressing this problem is to have a plurality of pins 118 or resilient contacts 220 per corresponding contact 114 or 214. The embodiment of FIGS. 4A and 4B, each arm of each horseshoe contact 318, acts largely as an independent contact, ensuring good connectivity.

In this manner, for a reduction in overall conductivity to occur in a signal path, at least two contact-to-contact paths would have to lose conductivity simultaneously. This amounts to at least two independent events, both of which are fairly rare. If for example, there was a 0.05 chance of either of two contact pairs falling below 50% of normal conductivity, then the chance of both falling below 50% at the same time would be 0.0025.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of performing a surgical procedure using an electrical surgical device, comprising:
(a) providing an electrical surgical assembly including said surgical device connected to a base station by a cable having a plurality of mutually electrically insulated conductors, and an electrical connector interposed at a point between said base station and said device;
(b) wherein said connector is comprised of a first half and a second half that, when connected together, have freedom of rotation relative to each other; and
(c) performing said surgical procedure and permitting said connector to permit said relative rotation thereby avoiding a problem of cable twisting.

2. The method of claim 1, wherein said first half of said connector is fixed into said base station.

3. The method of claim 1, wherein said surgical device is an angioplasty catheter.

4. The method of claim 1, wherein said surgical device is an electric scalpel.

5. The method of claim 1, wherein said connector said cable comprises a first cable portion and a second cable portion and wherein said connector is interposed between said first cable portion and said second cable portion.

6. An electrical medical device assembly, comprising:
(a) a handheld unit, adapted to be manipulated by a medical professional and requiring multi-conductor electrical connection to a base unit;
(b) a base unit adapted to provide electrical power to said handheld unit;
(c) a multi-conductor electrical cable connecting said handheld unit to said base unit; and
(d) a multi-conductor electrical connector interposed between said handheld unit and said base unit, said connector including a first half and a second half and wherein relative rotation is permitted between said first half and said second half.

7. The assembly of claim 6, wherein said first half of said connector is fixed into said base station.

8. The assembly of claim 6, wherein said surgical device is an angioplasty catheter.

9. The assembly of claim 6, wherein said surgical device is an electric scalpel.

10. The assembly of claim 6, wherein said cable comprises a first cable portion and a second cable portion and wherein said connector is interposed between said first cable portion and said second cable portion.

11. The assembly of claim 6, wherein said first half of said connector defines a set of contacts having circular conductive surfaces and wherein said second half of said connector includes a set of resilient contacts, each positioned to contact one said circular conductive surfaces to create an electrical connection.

12. The assembly of claim 11, wherein each of said circular conductive surfaces is contacted by a single resilient contact.

13. The assembly of claim 11, wherein said circular conductive surfaces are concentric and said resilient contacts are spring-loaded pins.

14. The assembly of claim 11, wherein said circular conductive surfaces are stacked and said resilient contacts are bent metal sheets.

15. The assembly of claim 13, wherein said second half of said connector clamps around said first half.

16. The assembly of claim 11, wherein said circular conductive surfaces are stacked and said resilient contacts are resilient horseshoe shaped elements.

17. A connector comprising:
(a) a first half that defines a plurality of contacts having circular conductive surfaces; and
(b) a second half that includes a set of resilient contacts, each positioned to contact one of said circular conductive surfaces to create an electrical connection.

18. The connector of claim 17, wherein said circular conductive surfaces are concentric and said resilient contacts are spring-loaded pins.

19. The connector of claim 17, wherein said circular conductive surfaces are stacked and said resilient contacts are bent metal sheets.

20. The connector of claim 19, wherein said second half of said connector clamps around said first half.

21. The connector of claim 17, wherein said circular conductive surfaces are stacked and said resilient contacts are resilient horseshoe shaped elements.
